(19) United States
(12) Patent Application Publication
(10) Pub. No.: US 2017/0336706 A1
(43) Pub. Date: Nov. 23, 2017

(54) X-RAY BIOPSY SPECIMEN IMAGER AND METHODS

(71) Applicant: LI-COR, INC., Lincoln, NE (US)

(72) Inventor: HAN-WEI WANG, Lincoln, NE (US)

(73) Assignee: LI-COR, INC., Lincoln, NE (US)

(21) Appl. No.: 15/590,631

(22) Filed: May 9, 2017

Related U.S. Application Data
(60) Provisional application No. 62/339,657, filed on May 20, 2016.

(51) Int. Cl. G03B 42/02; A61B 6/00; G01N 23/223; G01N 23/04

(52) U.S. Cl. CPC G03B 42/026; G01N 23/046; A61B 6/547; G01N 23/223; A61B 6/5205; A61B 6/545; G03B 42/02

(57) ABSTRACT

Methods and devices are disclosed for the tomographic imaging of a biological sample from almost all rotational perspectives in three-dimensional space and with multiple imaging modalities. A biological sample is positioned on an imaging stage that is capable of nearly full 360-degree rotation in at least one of two substantially orthogonal axes. Positioned about the stage is an X-ray imaging module enabling the recording of a series of images. A reflected light imaging module can also be positioned about the stage to enable recording of black and white or color white light images. A computer can use the images to construct three-dimensional models of the sample and to render images of the sample conveying information from one or more imaging channels.

### CROSS-REFERENCES TO RELATED APPLICATIONS

This application claims the benefit of U.S. Provisional Application No. 62/339,657, filed May 20, 2016, which is incorporated in its entirety herein for all purposes.

### STATEMENT AS TO RIGHTS TO INVENTIONS MADE UNDER FEDERALLY SPONSORED RESEARCH AND DEVELOPMENT

NOT APPLICABLE

### BACKGROUND

With growing research into visual methods for identifying cancer and other types of cells or tissues, image-guided applications will make a significant impact on surgical guidance and cancer margin detection. In particular, the assessment of tumor margin during surgery is essential to the optimal outcome of many oncologic procedures. Tumor margins are the healthy tissue surrounding the tumor, more specifically, the distance between the tumor tissue and the edge of the surrounding tissue removed along with the tumor. Ideally, the margins are selected so that the risk of leaving tumor tissue within the patient is low. These imaging devices are intended for use in the operating room, frozen room or permanent pathology lab to help in the examination of resected specimens to identify putative disease regions, alongside other standard localization methods such as palpation and inspection.

In many cancer surgeries deep surgical cavities with closed spaces and hidden linings pose significant challenges for the use of over-head imaging systems. This is particularly true for breast-conserving surgeries and treatments of head and neck cancers. Discharging bio-fluids and small fields of view also can compromise the utility of handheld fluorescence devices for margin assessment at the surgical cavity. Therefore, intraoperative diagnosis on resected surgical samples promises to be a more effective means for margin assessment in many surgical cancer treatment applications.

Tomographic imaging, such as by X-ray imaging, can either alone or in conjunction with reflected white light imaging provide surgeons with important visual data to help guide surgical procedures. X-ray imaging has been applied for other analytical applications. For example, X-rays microfocused to a spot size of less than 50 micrometers have been used to image integrated circuits at a distance of between 1 and 10 millimeters from the X-ray source (U.S. Pat. No. 7,215,736). X-ray analysis of small organisms has been performed using heavy metal staining and a multiple irradiation energies (U.S. patent application Ser. No. 14/354,855). Radiographs have been produced from dental X-ray imaging (U.S. Pat. No. 8,750,450). Mammography with in vivo breast biopsies have been performed with X-ray tomosynthesis (U.S. Pat. Nos. 6,375,352; 7,463,713; 8,041,094; 8,532,745; and 8,831,171). Other X-ray devices allow for the two-dimensional rotation of a sample (U.S. patent application Ser. No. 11/066,142), or of an X-ray source (U.S. Pat. No. 9,138,193). Surgical imaging capabilities can be significantly extended through the combination of X-ray imaging with visible light imaging, full three-dimensional rotation of resected tissue samples during imaging, and computer-assisted presentation of multiple images representative of the one or more imaging modalities.

### BRIEF SUMMARY

In general, provided herein are devices and methods for multi-axis 3D tomographic radiographic specimen imaging to provides a surgeon or pathologist with 3D rotational views of the sample from positions distributed about the sample in almost any three-dimensional rotational direction. The sample is positioned on an imaging stage that is transparent to X-rays, visible light, near-infrared light, or other types of radiation relevant to the imaging modalities being used. The imaging stage is rotatable in almost any direction, so that cameras, detectors, or sensors located at positions about the stage can record images of the sample taken from multiple angles. Because the imaging stage is transparent, these images of the sample can be recorded through the stage itself.

The devices and methods are used for X-ray imaging to visualize tissue density and radiopaque tissue inserts. The resulting multi-axis 3D rotational images can provide, for example, information to a surgeon or pathologist on the location of a breast clip or of mineralization in the case of a breast specimen. Other imaging modalities, such as full-color or black-and-white imaging of the sample with cameras that record reflected visible light, can also be recorded with the devices and methods. The rotational views can then provide visual information for the specimen of interest in both white light and X-ray channels.

A display device can be used to present images rendered using information recorded in each of the imaging modalities. The presentation can simultaneously or sequentially display images from multiple modalities or from multiple angles relative to the sample. This multimodal and multi-axis imaging can, for example, offer a novel way to visualize resected tissue samples, providing surgeons with an improved understanding of tumor outlines and tissue characteristics.

One provided apparatus for imaging a biological sample with X-rays comprises a rotatable imaging stage adapted for supporting at least a portion of a biological sample within an imaging volume. The rotatable imaging stage has a first rotational axis and a second rotational axis. The second rotational axis is substantially orthogonal to the first rotational axis. The apparatus further comprises an X-ray source configured to irradiate the imaging volume with X-rays and an X-ray imager configured to detect X-rays exiting the imaging volume.

“Substantially orthogonal” or “substantially perpendicular” axes include those that are 90° from one another in one or more planes or at or above 85°, 80°, 75°, 70°, 65°, 60°, or other non-zero value from one another as known in the art. Axes that are not exactly perpendicular (i.e., 90°) from one another may be off due to machining tolerances or purposely aligned with one another in order to reduce reflections, simplify mechanical design, or other reasons.

In some embodiments, the imaging stage comprises a transparent portion that is transparent to X-rays. In some embodiments, the transparent portion comprises glass or acrylic.

In some embodiments the imaging stage comprises a plurality of marks at predetermined intervals, wherein the marks comprise a radiopaque material. In some embodiments, the radiopaque material comprises a metal.

In some embodiments, the X-ray source is an X-ray tube. In some embodiments, the X-ray imager is a flat panel detector.

In some embodiments, the apparatus further comprises a computer processer operatively connected with a machine-readable non-transitory medium embodying information indicative of instructions for causing the computer processor to perform operations. In some embodiments, the computer processor records X-ray images of the biological sample using the X-ray imager. In some embodiments, the computer processor rotates the rotatable imaging stage around at least one of the first rotational axis and the second rotational axis.

In some embodiments, the computer processor constructs a three-dimensional X-ray model from two or more X-ray images, wherein each of the two or more X-ray images is recorded with the rotatable imaging stage oriented in different positions around at least one of the first rotational axis or the second rotational axis. In some embodiments, the computer processor renders an image produced from the X-ray model.

Also provided is an apparatus for imaging a biological sample with visible light and X-rays, comprises a rotatable imaging stage adapted for supporting at least a portion of a biological sample within an imaging volume. The rotatable imaging stage has a first rotational axis and a second rotational axis. The second rotational axis is substantially orthogonal to the first rotational axis. The apparatus further comprises an X-ray source configured to irradiate the imaging volume with X-rays, an X-ray imager configured to detect X-rays exiting the imaging volume, and a camera configured to have a depth of focus within the imaging volume and to detect reflected light.

In some embodiments, the imaging stage comprises a transparent portion that is transparent to visible light. In some embodiments, the transparent portion is transparent to X-rays. In some embodiments, the transparent portion comprises glass or acrylic. In some embodiments the imaging stage comprises a plurality of marks at predetermined intervals, wherein the marks comprise a radiopaque material. In some embodiments, the radiopaque material comprises a metal.

In some embodiments, the X-ray source is an X-ray tube. In some embodiments, the X-ray imager is a flat panel detector.

In some embodiments, the apparatus further comprises a computer processer operatively connected with a machine-readable non-transitory medium embodying information indicative of instructions for causing the computer processor to perform operations. In some embodiments, the computer processor records reflected light images of the biological sample using the camera. In some embodiments, the computer processor records X-ray images of the biological sample using the X-ray imager. In some embodiments, the computer processor rotates the rotatable imaging stage around at least one of the first rotational axis and the second rotational axis.

In some embodiments, the computer processor constructs a three-dimensional reflected light model from two or more reflected light images, wherein each of the two or more reflected light images is recorded with the rotatable imaging stage oriented in different positions around at least one of the first rotational axis and the second rotational axis. In some embodiments, the computer processor constructs a three-dimensional X-ray model from two or more X-ray images, wherein each of the two or more X-ray images is recorded with the rotatable imaging stage oriented in different positions around at least one of the first rotational axis or the second rotational axis. In some embodiments, the computer processor renders an image produced from the reflected light model and the X-ray model, wherein the reflected light model and the X-ray model are co-registered in three-dimensional space.

In some embodiments, the computer processer associates a first X-ray image of the X-ray images with a first reflected light image of the reflected light images based on angles of the first and second rotational axes. In some embodiments, the computer processor renders a combined image based on the first X-ray and first reflected light images. In some embodiments, the computer processor displays the combined image to a user in series with other combined images.

Also provided is a method for imaging a biological sample with X-rays. The method comprises irradiating a biological sample within an imaging volume on a rotatable imaging stage with X-rays using an X-ray source. The rotatable imaging stage has a first rotational axis, a second rotational axis, and a transparent portion. The second rotational axis is substantially orthogonal to the first rotational axis. The transparent portion is transparent to X-rays. The method further comprises recording, using an X-ray imager, a first X-ray image of the X-rays exiting the imaging volume. The method further comprises rotating the imaging stage by a predetermined amount around at least one of the first rotational axis and the second rotational axis. The method further comprises irradiating the biological sample with X-rays. The method further comprises recording a second X-ray image of the X-rays exiting the imaging volume through the transparent portion of the rotatable imaging stage.

In some embodiments, the method further comprises constructing a three-dimensional X-ray model from the first and second X-ray images using the computer. In some embodiments, the method further comprises rendering an image produced from the X-ray model.

Also provided is a method for imaging a biological sample with visible light and X-rays. The method comprises illuminating a biological sample within an imaging volume on a rotatable imaging stage with visible light. The rotatable imaging stage has a first rotational axis, a second rotational axis, and a transparent portion. The second rotational axis is substantially orthogonal to the first rotational axis. The transparent portion is transparent to visible light, near-infrared light, and X-rays. The method further comprises recording, using a camera, a first reflected light image of visible light reflected by the biological sample. The method further comprises irradiating the biological sample on the rotatable imaging stage with X-rays using an X-ray source. The method further comprises recording, using an X-ray imager, a first X-ray image of the X-rays exiting the imaging volume. The method further comprises rotating the imaging stage by a predetermined amount around at least one of the first rotational axis and the second rotational axis. The method further comprises recording a second reflected light image of visible light reflected by the biological sample through the transparent portion of the rotatable imaging stage. The method further comprises irradiating the biological sample with X-rays. The method further comprises recording a second X-ray image of the X-rays exiting the imaging volume through the transparent portion of the rotatable imaging stage.

In some embodiments, the method further comprises constructing a three-dimensional reflected light model from the first and second reflected light images using a computer. In some embodiments, the method further comprises constructing a three-dimensional X-ray model from the first and second X-ray images using the computer. In some embodiments, the method further comprises rendering an image produced from the reflected light model and the X-ray model, wherein the reflected light model and the X-ray model are co-registered in three-dimensional space.

In some embodiments, the method further comprises positioning the X-ray imager between the biological sample and the camera. In some embodiments, the X-ray imager is a flat panel detector. In some embodiments, the flat panel detector has a detection face and a display face, wherein the display face is opposite to the detection face, wherein the detection face is directed towards the biological sample, and wherein the display face is directed towards the camera. In some embodiments, the method further comprises irradiating the biological sample on the rotatable imaging stage with X-rays using an X-ray source, wherein the biological sample is positioned between the X-ray source and the flat panel detector, and wherein the X-ray source, the biological sample, the flat panel detector, and the camera are collinear. In some embodiments, the method further comprises converting the X-rays detected by the detection face of the flat panel detector into a first X-ray image displayed on the display face of the flat panel detector. In some embodiments, the method further comprises recording using the camera the first X-ray image displayed on the display face of the flat panel detector. In some embodiments, the method further comprises positioning the flat panel detector such that the flat panel detector is not between the biological sample and the camera. In some embodiments, the method further comprises rotating the imaging stage by a predetermined amount around at least of the first rotational axis and the second rotational axis. In some embodiments, the method further comprises positioning the flat panel detector between the biological sample and the camera. In some embodiments, the method further comprises irradiating the biological sample on the rotatable imaging stage with X-rays using an X-ray source. In some embodiments, the method further comprises converting the X-rays detected through the transparent portion of the rotatable imaging stage by the detection face of the flat panel detector into a second X-ray image displayed on the display face of the flat panel detector. In some embodiments, the method further comprises recording, using the camera, the second X-ray image displayed on the display face of the X-ray flat panel detector.

### BRIEF DESCRIPTION OF THE DRAWINGS

FIG. 1 is a perspective illustration of a rotatable stage and X-ray imaging system in accordance with an embodiment.

FIG. 2 is a perspective illustration of a rotatable stage an X-ray imaging system and a reflected light imaging system in accordance with an embodiment.

FIG. 3 is a flowchart of a process in accordance with an embodiment.

FIG. 4 is a flowchart of a process in accordance with an embodiment.

FIG. 5 is a perspective illustration of an imaging system with X-ray flat panel detector in accordance with an embodiment.

### DETAILED DESCRIPTION

The present invention relates in part to multi-axis three-dimensional X-ray imaging devices and methods for visualizing samples. The devices and methods can be used to record and display X-ray images of a biological sample representing views of the sample from almost any position rotated about the sample in three-dimensional space. The devices and methods can also be used to record and display reflected light images of the sample representing views from the same three-dimensional positions as those used to record the X-ray images.

A technical advantage of the embodiments described herein is that a surgeon can have enhanced access to visualized information regarding the location and characteristics of diseased and healthy cells and tissue within a resected biopsy sample. This can allow a surgeon to more accurately assess tumor margins during surgical procedures, which can in turn increase the probability of a successful surgery and the survival rate of the patient.

By combining multi-axis rotation three-dimensional imaging with X-ray and reflected light modalities, the inventors have made the surprising discovery of a novel way to look at a resected tissue sample. For example, by combining reflected visible light imaging with an X-ray imaging module, one embodiment of the system describe herein provides three-dimensional full-rotation surface mapping together with X-ray projections from different angles. This can give a surgeon a unique and comprehensive understanding of a tumor tissue from the optical channel together with tomographic information from the X-ray channel. The optical channel gives the outline of the tissue, and the X-ray channel shows the tissue density and projection, as well as information about any metal or wire inserts placed inside the tissue.

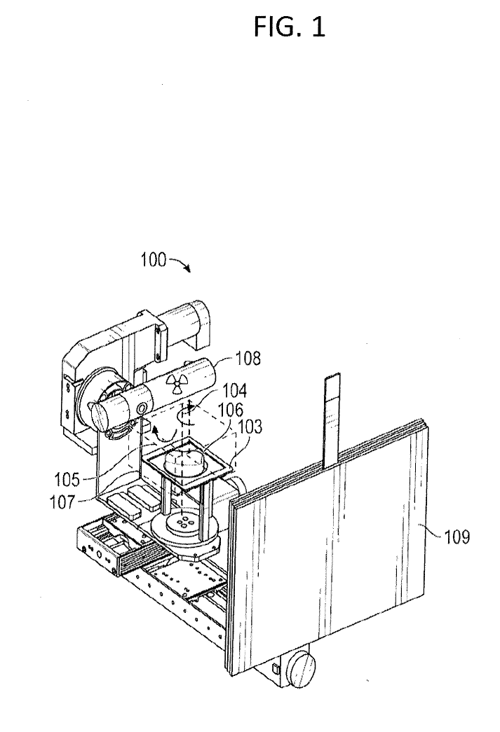

FIG. 1 illustrates one embodiment as a descriptive example. Shown is an apparatus 100 having an imaging stage 103 that has a first rotational axis 104 and a second rotational axis 105. A biological sample 106 is shown being supported by the imaging stage 103. The biological sample 106 is within an imaging volume 107 proximate to the imaging stage 103. An X-ray source 108 is configured to irradiate the imaging volume 107 with X-rays, and an X-ray imager 109 is configured to detect X-rays exiting the imaging volume 107.

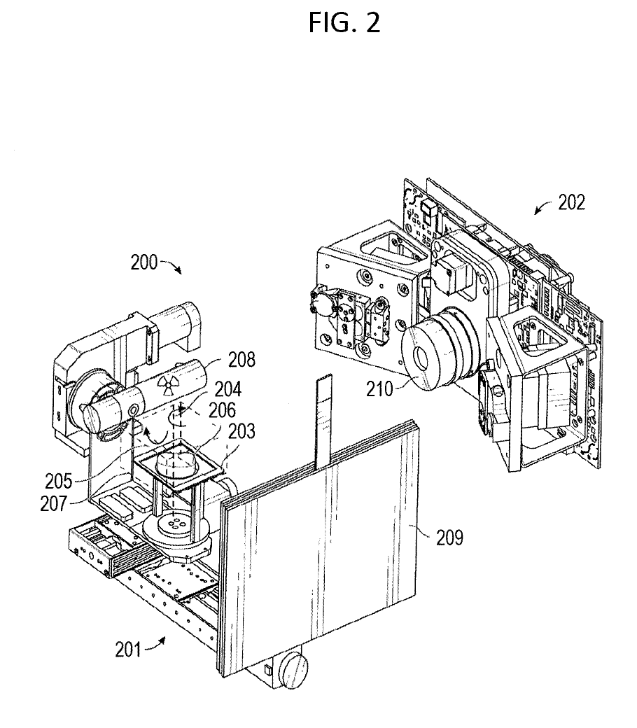

FIG. 2 illustrates another embodiment as a descriptive example. Shown is an apparatus 200 comprising a sample positioning module 201 and an optical imaging module 202. The sample positioning module has an imaging stage 203 that has a first rotational axis 204 and a second rotational axis 205. A biological sample 206 is shown being supported by the imaging stage 203. The biological sample 206 is within an imaging volume 207 proximate to the imaging stage 203. An X-ray source 208 is configured to irradiate the imaging volume 207 with X-rays, and an X-ray imager 209 is configured to detect X-rays exiting the imaging volume 207. The optical imaging module 202 has a camera 210 configured to have a depth of focus within the imaging volume.

The biological sample can comprise material removed from a subject. The subject is typically a human, but also can be another animal. The subject can be, for example, rodent, canine, feline, equine, ovine, porcine, or another primate. The subject can be a patient suffering from a disease. In some embodiments, the subject is a cancer patient. In certain aspects, the biological sample comprises a tumor, such as tumor tissue or cells. In certain aspects, the biological sample comprises a peripheral biopsy of a tissue sample previously removed. In another aspect, the biological sample is tumor tissue such as a breast core biopsy. The biological sample size can be as small as a tissue slice.

The rotatable imaging stage supporting the biological sample is equipped with rotational motors and stages to control the view angle and position of a sample within the imaging volume. By rotating a sample in two degrees of freedom, the stage can allow an imager to efficiently provide a full-rotation three-dimensional image. A first rotational axis can, for example, provide 360-degree movement along the z-axis (roll) relative to the sample. A second rotational axis can, for example, tilt along the y-axis (pitch) for imaging at different perspectives. Tilting of the sample stage also allows projection views from the top and the bottom of the sample via a transparent window. In some embodiments, the rotational imaging stage can also be moved in an X-Y plane to allow for the registration of the sample to the center of the imaging volume.

Rotational combinations can allow the entire sample to be imaged. To collect pertinent imaging projections along a sample for subsequent three-dimensional reconstruction, the rotational imaging stage can rotate the object in rolling and tilting degrees of freedom. In some embodiments, to provide comprehensive coverage of sample features the rolling angle is in the range of from 7.5 degrees to 45 degrees, depending on the complexity of the sample. In some embodiments, a rolling step of 22.5 degrees and a tilting angle of ±35 degrees offers a full rotation for three-dimensional inspection and imaging of the sample.

Rotation of the imaging stage around one or both of the first and second rotational axis can be accomplished through the use of rotor bearings connected to the stage. A rotor bearing can be a hub, axle, or other mechanical element that bears contact between at least two parts and that allows for rotation around an axis. A rotary bearing can include circular tracks and cages for ball bearings, lubricant surfaces, and other friction-reducing implements.

The imaging volume is defined as the volume formed by the fields of illumination or other electromagnetic radiation, by the depth-of-focus of an object lens, and by the field-of-view of an imaging head. The imaging volume is typically configured such that all cameras, detectors, sensors, and other image capturing elements of the apparatus are tolerant of placement of the sample anywhere within the volume.

The X-ray source can be any artificial X-ray source configured to irradiate the imaging volume with X-rays. In some embodiments, the X-ray source is an X-ray tube. The X-ray tube can comprise a rotating anode tube. In some embodiments, the X-ray source is a solid-anode microfocus X-ray tube or a metal-jet-anode microfocus X-ray tube.

The X-ray imager can be any device configured to measure the properties of X-rays exiting the image volume. The X-ray imager can comprise, for example, one or more of a sensitized photographic plate, sensitized photographic film, a photostimulable phosphor plate, a semiconductor or solid state detector, or a scintillator. In some embodiments, the X-ray imager comprises a scintillator. The scintillator can comprise any material that converts an X-ray photon to a visible light photon. The scintillator can comprise one or more organic or inorganic compounds. The scintillator compounds can comprise, for example, barium fluoride, calcium fluoride doped with europium, bismuth germinate, cadmium tungstate, cesium iodide doped with thallium, cesium iodide doped with sodium, undoped cesium iodide, gadolinium oxysulfide, lanthanum bromide doped with cerium, lanthanum chloride doped with cerium, lead tungstate, lutetium iodide, lutetium oxyorthosilicate, sodium iodide doped with thallium, yttrium aluminum garnet, zinc sulfide, or zinc tungstate. In some embodiments, the scintillator comprises sodium iodide, gadolinium oxysulfide, or cesium iodide.

In some embodiments, the X-ray imager is a an X-ray flat panel detector. The flat panel detector can comprise a scintillator material and a photodiode transistor array. The flat panel detector can further comprise one or more readout circuits. The flat panel detector can comprise a detection face and a display face on opposite sides of the detector from one another. The detection face can be directed towards the biological sample and the X-ray source so as to be contacted with X-rays generated by the X-ray source and passing through the imaging volume. The display face can be directed towards a camera so that an X-ray image displayed on the display face can be recorded using the camera. In some embodiments, the X-ray image is displayed on the display face by generating visible light that is recorded by a visible light camera configured to have a depth of focus that corresponds to the distance between the display face and the camera.

In preferred embodiments, the X-ray source, biological sample, and X-ray imager are collinear with one another. In this configuration, the X-ray imager can record information related to X-rays that are generated by the X-ray source, travel through the imaging volume, and contact the sensors of the X-ray imager. As the X-rays travel through the imaging volume, they can be affected by the properties of any material, such as a biological sample, within the imaging volume. Regions of the biological sample with differing degrees of radiodensity will permit differing amounts of X-rays to pass through those regions. These differing amounts will result in changes in the signal intensities detected by different areas of the X-ray imager sensors. As the rotatable imaging stage is moved around one or both of its rotational axes, the locations of any radiodense regions of the biological sample relative to the locations of the X-ray source and X-ray imager will be changed. This allows for the recording of X-ray images with the X-ray imager that provide information about the radiopacity of the sample as detected from multiple perspectives.

Illumination sources can be mounted proximate to the imaging volume in order to illuminate the sample with white light, monochrome light, near-infrared light, fluorescence light, or other electromagnetic radiation. One or more white lights can be used to illuminate the imaging volume. In some embodiments, the illumination of the biological sample with visible light is performed at one or more wavelengths of about 380 nm to about 700 nm. These wavelengths include, for example, about 380, 390, 400, 425, 450, 475, 500, 525, 550, 575, 600, 625, 650, 675, or about 700 nm. These wavelengths can occur in combination, such as in broadband white light.

One or more cameras of the apparatus can have an actively or passively cooled heat exchanger to maintain imaging sensors at low temperatures. The imaging sensors can be charge coupled device imaging sensors. The cooling can prevent optical background noise such as darkness or blooming. Other approaches for improving camera sensitivity to compensate for low light levels of fluorescence can include imaging with a monochrome sensor, long exposure durations, and electronic noise suppression methods. Exemplary camera and optical components are described in U.S. Pat. Nos. 7,286,232, 8,220,415, and 8,851,017.

The rotatable imaging stage can comprise a transparent portion, such as a window. The window can be transparent at the working wavelengths for X-rays. The transparent portion can further be transparent to reflected light. To accommodate a large size sample, the window can be configured to a shape that is wider than either the projection size of the imaging volume or the footprint of the target sample. A circle on the window can be used to mark the border of a suggested imaging area.

The material of the transparent portion can be, for example, borosilicate-based glass, acrylic, or other transparent material. The surface could be treated or coated for optical or surface functional requirements. Non-limiting examples of these treatments include those providing anti-reflection, transparency, absorption, hydrophobic, or hydrophilic properties to the surface.

The rotatable imaging stage can further comprise one or more marks. The marks can be regularly spaced or irregularly spaced. The marks can be configured to provide reference scales to users of the apparatus. The marks can also provide references to a computer processor used to analyze and manipulate images recorded of the sample within the imaging volume. In some embodiments, the marks comprise a radiopaque material. The radiopaque material can comprise a polymer or a metal.

The devices and methods can utilize a computing apparatus that is programmed or otherwise configured to automate and/or regulate one or more steps of the methods or features of the devices provided herein. Some embodiments provide machine executable code in a non-transitory storage medium that, when executed by a computing apparatus, implements any of the methods or operates any of the devices described herein. In some embodiments, the computing apparatus operates the power source and/or pump control.

In some embodiments, the apparatus comprises a computer processor that can record images of the biological sample. The recorded images can be reflected light images captured by a camera configured to detect reflected light. In some embodiments, the reflected light is visible light. The recorded images can be X-ray images captured by an X-ray imager. The X-ray images can be captured by a camera configured to detect light images presented on a display face of an X-ray flat panel detector. The computer processor can tag the recorded images with information related to the relative positions of one or more of cameras, imagers, detectors, or sensors, with respect to the rotatable imaging stage. The computer process can tag the recorded images with information related to the rotational position of the biological sample around either or both of a first and second rotational axes. The locational and positional tags can use information determined by detecting the locations and orientations of one or more marks on the rotational imaging stage.

In some embodiments, the computer processor can control the rotation of the rotatable imaging stage. The rotation can be about one or both of the first and second rotational axes. The rotation can occur simultaneously along with image recording. The rotation can be stopped during image recording. In some embodiments, the rotation is from one predetermined position to another. In some embodiments, the rotation is to a series of multiple different predetermined positions. The computer can record images captured in one or more channels or modalities at each position. As a non-limiting example, the computer can capture a reflected light image and an X-ray image at each position that the rotatable imaging stage is moved to. The computer processor can rotate the imaging stage so that a transparent portion of the imaging stage is between the sample and one or more cameras, imagers, detectors, or sensors. Images or other information can then be recorded of the sample through the transparent portion of the imaging stage.

In some embodiments, the computer processer can construct models based on the recorded images. The models can be three-dimensional models. The models can comprise series of discrete images, each recorded as the rotatable imaging stage was at a different orientation relative to the apparatus element used in recording the images. The models can further comprise images constructed by interpolating information contained in discrete images. In some embodiments, the models are wireframe models created by translating two or more images into a polygonal mesh. The models can comprise surface information about the biological subject. The models can comprise tomographic information about the biological subject.

In some embodiments, the computer processer can render images produced from the constructed models. The rendered images can be identical to images recorded using the cameras, imagers, detectors, or sensors. The rendered images can be constructions based on information in the recorded images. The rendered images can contain images or information collected with one channel or modality. The rendered images can overlay images or information collected with two or more channels or modalities. As a non-limiting example, a rendered image can overlay reflected light information showing a visible light view of the biological sample and X-ray information showing locations of radiodense regions within the biological sample. Typically, when a rendered image overlays images or information from multiple channels, modalities, or models, the models are co-registered in three-dimensional space so that the image presents information for each modality as seen from a single viewpoint.

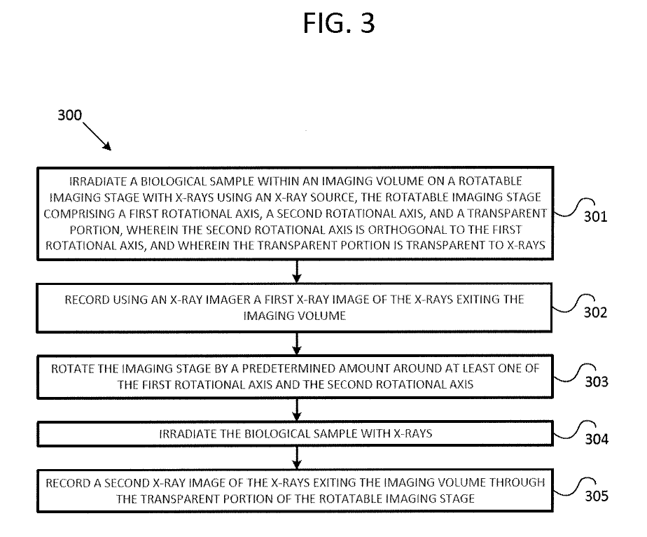

FIG. 3 presents a flowchart of a process 300 for imaging a biological sample with X-rays. In operation 301, a biological sample within an imaging volume on a rotatable imaging stage irradiated with X-rays using an X-ray source, the rotatable imaging stage comprising a first rotational axis, a second rotational axis, and a transparent portion, wherein the second rotational axis is substantially orthogonal to the first rotational axis, and wherein the transparent portion is transparent to X-rays. In operation 302, a first X-ray image of the X-rays exiting the imaging volume is recorded using an X-ray imager. In operation 303, the imaging stage is rotated by a predetermined amount around at least one of the first rotational axis and the second rotational axis. In operation 304, the biological sample is irradiated with X-rays. In operation 305, a second X-ray image of the X-rays exiting the imaging volume through the transparent portion of the rotatable imaging stage is recorded.

In some embodiments, the method further comprises an operation to construct a three-dimensional X-ray model from the first and second X-ray images using a computer. In some embodiments, the method further comprises an operation to render an image produced from the X-ray model.

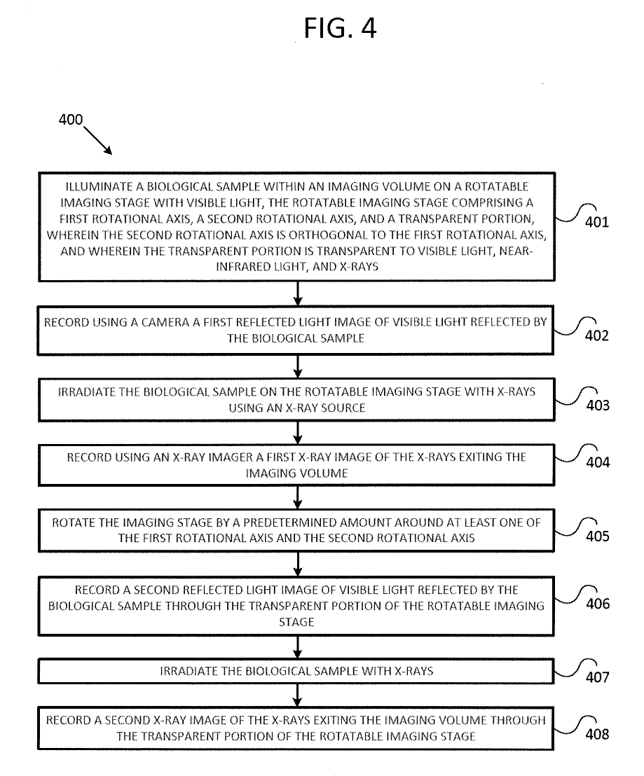

FIG. 4 presents a flowchart of a process 400 for imaging a biological sample with reflected visible light and X-rays. In operation 401, a biological sample within an imaging volume on a rotatable imaging stage is illuminated with visible light, the rotatable imaging stage comprising a first rotational axis, a second rotational axis, and a transparent portion, wherein the second rotational axis is substantially orthogonal to the first rotational axis, and wherein the transparent portion is transparent to visible light, near-infrared light, and x-rays. In operation 402, a first reflected light image of visible light reflected by the biological sample is recorded using a camera. In operation 403, the biological sample on the rotatable imaging stage is irradiated with X-rays using an X-ray source. In operation 404, a first X-ray image of the X-rays exiting the imaging volume is recorded using an X-ray imager. In operation 405, the imaging stage is rotated by a predetermined amount around at least one of the first rotational axis and the second rotational axis. In operation 406, a second reflected light image of visible light reflected by the biological sample through the transparent portion of the rotatable imaging stage is recorded. In operation 407, the biological sample is irradiated with X-rays. In operation 408, a second X-ray image of the X-rays exiting the imaging volume through the transparent portion of the rotatable imaging stage is recorded.

The process presented in FIG. 4 can be carried out with an apparatus similar or identical to the one presented in FIG. 2. In some embodiments, the X-ray source 208 and the X-ray imager 209 are placed orthogonal to the optical imaging module 202. The center of the field-of-view of the X-ray imager 209 is on the same x-y plane as the field-of-view of the optical imaging module 202. In some embodiments, the X-ray source 208 and the X-ray imager 209 are placed at a defined angle along the z-axis of the imaging volume while maintaining the center of the field-of-view of the X-ray imager on the same x-y plane as the field-of-view of the optical imaging module 202. Therefore, if the optical module 202 is imaging the biological sample 206 at an angle of 0 degrees around the rotational axis of the imaging volume, the X-ray module can be used to record X-ray projection images of the biological sample at an angle of 90 degrees, 270 degrees, or any other defined angle around the rotational axis of the imaging volume. After the rotatable imaging stage has been rotated to a new orientation, and images of the biological sample have been recorded at this new orientation, registration of the images recorded with two or more modalities can be performed to provide co-localized and co-registered image information.

The process presented in FIG. 4 can be carried out with an apparatus in which the X-ray source 208 can irradiate the imaging volume with X-rays from the same direction as the direction of the optical module 202. In this case, the X-ray projection image recorded using the X-ray imager 209 is a view of the biological sample 206 from approximately the same angle as that of the image recorded using the optical module 202. In this embodiment, surface mapping images recorded using the optical module 202 will have registrations approximately identical to tomography images simultaneously recorded using the X-ray module. As a result, an operation involving subsequent co-localization and co-registration of the images recorded using different modalities can be eliminated.

In some embodiments, the method further comprises an operation to position the X-ray imager between the biological sample and the camera, wherein the X-ray imager is a flat panel detector, wherein the flat panel detector has a detection face and a display face, wherein the display face is opposite to the detection face, wherein the detection face is directed towards the biological sample, and wherein the display face is directed towards the camera. In some embodiments, the method further comprises an operation to irradiate the biological sample on the rotatable imaging stage with X-rays using an X-ray source, wherein the biological sample is positioned between the X-ray source and the flat panel detector, and wherein the X-ray source, the biological sample, the flat panel detector, and the camera are collinear. In some embodiments, the method further comprises an operation to convert the X-rays detected by the detection face of the flat panel detector into a first X-ray image displayed on the display face of the flat panel detector. In some embodiments, the method further comprises an operation to record using the camera the first X-ray image displayed on the display face of the flat panel detector. In some embodiments, the method further comprises an operation to position the flat panel detector such that the flat panel detector is not between the biological sample and the camera. In some embodiments, the method further comprises an operation to rotate the imaging stage by a predetermined amount around at least of the first rotational axis and the second rotational axis. In some embodiments, the method further comprises an operation to position the flat panel detector between the biological sample and the camera. In some embodiments, the method further comprises an operation to irradiate the biological sample on the rotatable imaging stage with X-rays using an X-ray source. In some embodiments, the method further comprises an operation to converting the X-rays detected through the transparent portion of the rotatable imaging stage by the detection face of the flat panel detector into a second X-ray image displayed on the display face of the flat panel detector. In some embodiments, the method further comprises an operation to record using the camera the second X-ray image displayed on the display face of the X-ray flat panel detector.

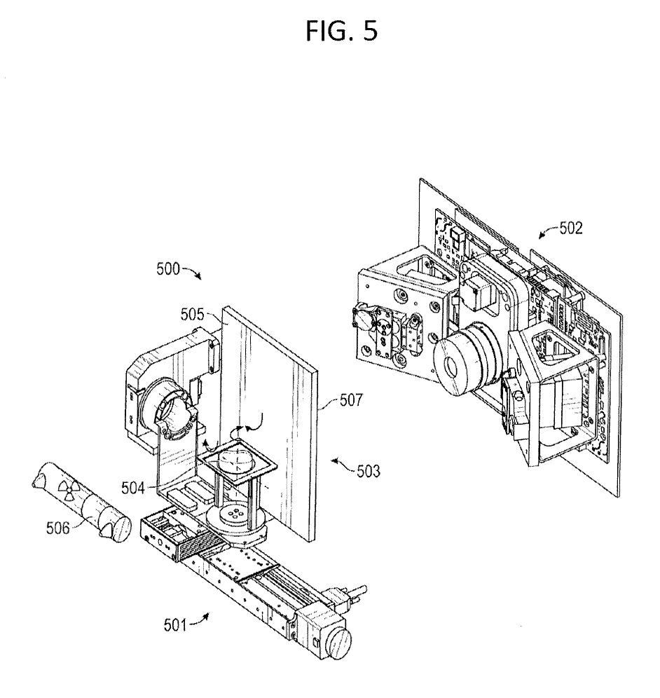

FIG. 5 illustrates one embodiment as a descriptive example. Shown is an apparatus 500 comprising a sample positioning module 501 and an optical imaging module 502. An X-ray flat panel detector 503 is positioned between a biological sample 504 and the optical imaging module 502. A detection face 505 of the flat panel detector 503 is contacted by X-rays generated by an X-ray source 506 that are not partially or completely blocked by regions of the biological sample 504. The flat panel detector 503 converts the X-rays detected by the detection face 505 into an X-ray image displayed on the display face 507 of the flat panel detector. The detection face 505 and the display face 507 are on opposite sides of the flat panel detector 503. The optical imaging module 502 can then be used to record the X-ray image displayed on the display face 503. To record images of the biological sample 504 with the optical imaging module 502 using reflected light, the flat panel detector 503 is repositioned so that it is not between the biological sample and the optical imaging module. In this way, the optical imaging module 502 can record images using a reflected visible light channel. The flat panel detector 503 can be repeatedly moved from a first position enabling recording of X-ray images with the optical imaging module 502 as described, to a second position enabling recording of non-X-ray images with the optical imaging module 302

The terms “about” and “approximately equal” are used herein to modify a numerical value and indicate a defined range around that value. If “X” is the value, “about X” or “approximately equal to X” generally indicates a value from 0.90X to 1.10X. Any reference to “about X” indicates at least the values X, 0.90X, 0.91X, 0.92X, 0.93X, 0.94X, 0.95X, 0.96X, 0.97X, 0.98X, 0.99X, 1.01X, 1.02X, 1.03X, 1.04X, 1.05X, 1.06X, 1.07X, 1.08X, 1.09X, and 1.10X. Thus, “about X” is intended to disclose, e.g., “0.98X.” When “about” is applied to the beginning of a numerical range, it applies to both ends of the range. Thus, “from about 6 to 8.5” is equivalent to “from about 6 to about 8.5.” When “about” is applied to the first value of a set of values, it applies to all values in that set. Thus, “about 7, 9, or 11%” is equivalent to “about 7%, about 9%, or about 11%.”

Systems that incorporate the apparatus are also provided. Systems can include, for example, power supplies, power regulators, and other elements enabling the operation of the apparatus. It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications, websites, and databases cited herein are hereby incorporated by reference in their entireties for all purposes.

1. An apparatus for imaging a biological sample, the apparatus comprising:
a rotatable imaging stage adapted for supporting at least a portion of a biological sample within an imaging volume, the rotatable imaging stage comprising a first rotational axis and a second rotational axis, wherein the second rotational axis is substantially orthogonal to the first rotational axis;
an X-ray source configured to irradiate the imaging volume with X-rays; and
an X-ray imager configured to detect X-rays exiting the imaging volume.

2. The apparatus of claim 1, wherein the imaging stage comprises a transparent portion that is transparent to X-rays.

3. The apparatus of claim 2, wherein the transparent portion comprises glass or acrylic.

4. The apparatus of claim 1, wherein the imaging stage comprises a plurality of marks at predetermined intervals, wherein the marks comprise a radiopaque material.

5. (canceled)

6. The apparatus of claim 1, wherein the X-ray source is an X-ray tube.

7. The apparatus of claim 1, wherein the X-ray imager is a flat panel detector.

8. The apparatus of claim 1, further comprising
a computer processer operatively connected with a machine-readable non-transitory medium embodying information indicative of instructions for causing the computer processor to perform operations comprising:
recording X-ray images of the biological sample using the X-ray imager; and
rotating the rotatable imaging stage around at least one of the first rotational axis and the second rotational axis.

9. The apparatus of claim 8, wherein the operations further comprise:
constructing a three-dimensional X-ray model from two or more X-ray images, wherein each of the two or more X-ray images is recorded with the rotatable imaging stage oriented in different positions around at least one of the first rotational axis or the second rotational axis; and
rendering an image produced from the X-ray model.

10. An apparatus for imaging a biological sample, the apparatus comprising:
a rotatable imaging stage adapted for supporting at least a portion of a biological sample within an imaging volume, the rotatable imaging stage comprising a first rotational axis and a second rotational axis, wherein the second rotational axis is substantially orthogonal to the first rotational axis;
an X-ray source configured to irradiate the imaging volume with X-rays;
an X-ray imager configured to detect X-rays exiting the imaging volume; and
a camera configured to have a depth of focus within the imaging volume and to detect reflected light.

11. The apparatus of claim 10, wherein the imaging stage comprises a transparent portion that is transparent to visible light.

12. The apparatus of claim 11, wherein the transparent portion is transparent to X-rays.

13. The apparatus of claim 12, wherein the transparent portion comprises glass or acrylic.

14. The apparatus of claim 10, wherein the imaging stage comprises a plurality of marks at predetermined intervals, wherein the marks comprise a radiopaque material.

15. (canceled)

16. The apparatus of claim 10, wherein the X-ray source is an X-ray tube.

17. The apparatus of claim 10, wherein the X-ray imager is a flat panel detector.

18. The apparatus of claim 10, further comprising
a computer processer operatively connected with a machine-readable non-transitory medium embodying information indicative of instructions for causing the computer processor to perform operations comprising:
recording reflected light images of the biological sample using the camera;
recording X-ray images of the biological sample using the X-ray imager; and
rotating the rotatable imaging stage around at least one of the first rotational axis and the second rotational axis.

19. The apparatus of claim 18, wherein the operations further comprise:
constructing a three-dimensional reflected light model from two or more reflected light images, wherein each of the two or more reflected light images is recorded with the rotatable imaging stage oriented in different positions around at least one of the first rotational axis and the second rotational axis;
constructing a three-dimensional X-ray model from two or more X-ray images, wherein each of the two or more X-ray images is recorded with the rotatable imaging stage oriented in different positions around at least one of the first rotational axis or the second rotational axis; and
rendering an image produced from the reflected light model and the X-ray model, wherein the reflected light model and the X-ray model are co-registered in three-dimensional space.

20. The apparatus of claim 18, wherein the operations further comprise:
associating a first X-ray image of the X-ray images with a first reflected light image of the reflected light images based on angles of the first and second rotational axes;
rendering a combined image based on the first X-ray and first reflected light images; and
displaying the combined image to a user in series with other combined images.

21. A method for imaging a biological sample, the method comprising:
irradiating a biological sample within an imaging volume on a rotatable imaging stage with X-rays using an X-ray source, the rotatable imaging stage comprising a first rotational axis, a second rotational axis, and a transparent portion, wherein the second rotational axis is substantially orthogonal to the first rotational axis, and wherein the transparent portion is transparent to X-rays;
recording using an X-ray imager a first X-ray image of the X-rays exiting the imaging volume;
rotating the imaging stage by a predetermined amount around at least one of the first rotational axis and the second rotational axis;
irradiating the biological sample with X-rays; and
recording a second X-ray image of the X-rays exiting the imaging volume through the transparent portion of the rotatable imaging stage.

22. The method of claim 21 further comprising:
constructing a three-dimensional X-ray model from the first and second X-ray images using the computer; and
rendering an image produced from the X-ray model.

23-25. (canceled)
